(19) United States
(12) Patent Application Publication
(10) Pub. No.: US 2010/0152763 A1
(43) Pub. Date: Jun. 17, 2010

(54) WIRELESS MAGNETIC NEEDLE SYSTEM FOR TRADITIONAL ORIENTAL MEDICINE

(75) Inventors: Yark Yeon Kim, Daejeon (KR); Won Ick Jang, Daejeon (KR); Chang Ho Bae, Daejeon (KR); Seon Hee Park, Daejeon (KR); Han Young, Yu (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon (KR)

(21) Appl. No.: 12/473,838

(22) Filed: May 28, 2009

(30) Foreign Application Priority Data
Dec. 16, 2008 (KR) 10-2008-0127752

(51) Int. Cl. A61B 17/34; A61N 2/00

(52) U.S. Cl. 606/189; 600/12

(57) ABSTRACT

A wireless magnetic needle system for traditional oriental medicine can apply a magnetic field to a needle being used for the acupuncture process on a patient by use of a Helmholtz coil capable of applying a magnetic field to a wide space and can apply a magnetic stimulus to the patient through the needle, thereby making it possible to maximize the therapy effect of the needle. The wireless magnetic needle system includes a current generating unit and a Helmholtz coil. The Helmholtz coil includes a first coaxial coil disposed over a bed for a patient, and a second coaxial coil disposed under the bed. The current generating unit generates a current, and the Helmholtz coil creates a magnetic field by using the current generated by the current generating unit.

### CROSS-REFERENCE TO RELATED APPLICATIONS

This application claims the priority of Korean Patent Application No. 2008-127752 filed on Dec. 16, 2008, in the Korean Intellectual Property Office, the disclosure of which is incorporated herein by reference.

### BACKGROUND OF THE INVENTION

1. Field of the Invention

The present invention relates to a wireless magnetic needle system for traditional oriental medicine, and more particularly, to a wireless magnetic needle system for traditional oriental medicine, which can apply a magnetic field to a needle being used on a patient for acupuncture by use of a Helmholtz coil capable of applying a magnetic field to a wide space and can apply a magnetic stimulus to the patient through the needle, thereby making it possible to maximize the therapeutic effect of the needle.

2. Description of the Related Art

In oriental medical science, an area of knowledge that has a history of several thousands of years, acupuncture needle points (acupuncture points and meridians) have been thought to serve as paths transmitting the energy of the human body. Thus, oriental medical science has made many efforts to cure the diseases of human bodies from the viewpoint of energy circulation (transmission). In this respect, acupuncture needle points serving as focal points of energy circulation are very important.

Thus, the practice of acupuncture has excellent effects on diseases such as chronic diseases that are not easily solved by the modern medical science. As a result of this, the U.S. Food and Drug Administration (USFDA), which had hitherto been conservative in alternative medicine, approved acupuncture as a therapeutic treatment in 1997.

Apparently, acupuncture is very effective, in that it can cure various diseases by stimulating specific portions (acupuncture needle points) of the human body. However, the core contents of acupuncture stimuli have, at times, been called into doubt.

Examples of stimuli applied to specific portions of the human body are a thermal stimulus, obtained by applying heat to a needle, an electrical stimulus obtained by using an electric needle inducing a micro current by applying electrical energy thereto, and a magnetic stimulus.

Examples of a magnetic needle inducing a magnetic stimulus are a metal needle having a permanent magnet to apply a magnetic field and a magnetic needle formed of a permanent magnet to apply a magnetic stimulus.

However, these conventional methods are very complex in terms of the structure and form of the needle. Also, the conventional methods are not economical to use in a disposable manner. Moreover, the conventional methods have limitations in the application of a magnetic stimulus to an acupuncture needle point.

### SUMMARY OF THE INVENTION

An aspect of the present invention provides a wireless magnetic needle system for traditional oriental medicine, which can apply a magnetic stimulus to an acupuncture needle point more efficiently by applying a magnetic stimulus to a wireless magnetic needle during the acupuncture process as well as by use of a wireless magnetic needle formed of a soft-magnetic material.

Another aspect of the present invention provides a wireless magnetic needle system for traditional oriental medicine, which can control a magnetic field statically or dynamically in order to diversify a magnetic stimulus applied to an acupuncture needle point.

According to an aspect of the present invention, there is provided a wireless magnetic needle system for traditional oriental medicine, including: a current generating unit generating a current; and a Helmholtz coil creating a magnetic field by using the current generated by the current generating unit, the Helmholtz coil including a first coaxial coil disposed over a bed for a patient; and a second coaxial coil disposed under the bed.

Herein, the wireless magnetic needle system may further include: a modulator modulating the current generated by the current generating unit; and an amplifier amplifying the current modulated by the modulator.

Also, the current generated by the current generating unit may have a waveform of one of a rectangular pulse wave, a sine wave and a sawtooth wave.

Also, the first coaxial coil or the second coaxial coil of the Helmholtz coil may have a quadrangular shape or a circular shape. The first coaxial coil of the Helmholtz coil may be movable and the second coaxial coil of the Helmholtz coil may be fixed. Both of the first coaxial coil and the second coaxial coil of the Helmholtz coil may be movable.

According to another aspect of the present invention, there is provided a wireless magnetic needle for a wireless magnetic needle system for traditional oriental medicine that applies a current generated by a current generating unit to a Helmholtz coil to apply a magnetic stimulus to a needle being used for the acupuncture process on a patient, wherein the wireless magnetic needle is formed of a soft-magnetic material.

Herein, the wireless magnetic needle may be formed of an alloy including iron, nickel or cobalt.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic view showing a wireless magnetic needle system for traditional oriental medicine according to an exemplary embodiment of the present invention;

FIG. 2 is a schematic view showing a wireless magnetic needle system for traditional oriental medicine according to another exemplary embodiment of the present invention;

FIGS. 3A and 3B are schematic views showing the structures of a Helmholtz coil according to exemplary embodiments of the present invention;

FIGS. 4A and 4B are waveform diagrams illustrating various forms of currents applied to a Helmholtz coil according to the present invention;

FIGS. 5A and 5B are graphs illustrating the operational characteristics of a soft-magnetic needle for traditional oriental medicine; and

FIGS. 6A to 6D are graphs illustrating the magnetic characteristics of a soft-magnetic needle for traditional oriental medicine.

### DETAILED DESCRIPTION OF THE PREFERRED EMBODIMENT

Exemplary embodiments of the present invention will now be described in detail with reference to the accompanying drawings. In the following description, detailed descriptions about well-known functions or structures will be omitted if they are deemed to obscure the subject matter of the present invention.

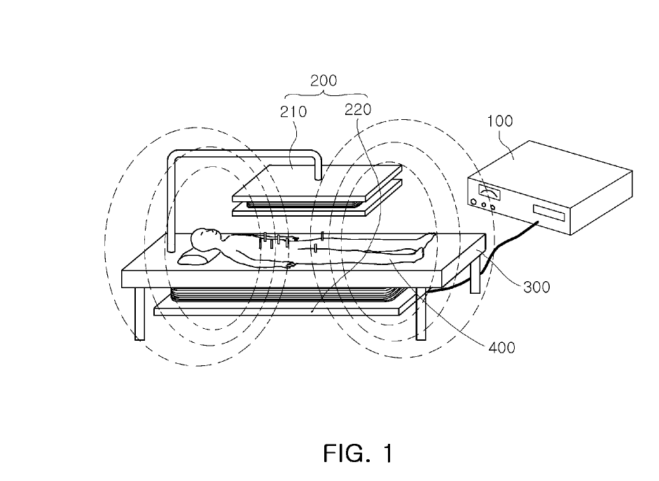

FIG. 1 is a schematic view showing a wireless magnetic needle system for traditional oriental medicine according to an exemplary embodiment of the present invention.

Referring to FIG. 1, a wireless magnetic needle system for traditional oriental medicine includes a current generating unit 100, a Helmholtz coil 200, and a bed 300, wherein the Helmholtz coil 200 includes a first coaxial coil 210 and a second coaxial coil 220. Herein, the wireless magnetic needle may be a soft-magnetic needle.

A patient 400, who is to be medically treated by the wireless magnetic needle system, is given acupuncture lying on the bed 300. Acupuncture is a medical art implemented in a general oriental medicine clinic, and thus a detailed description thereof is not provided herein. Thus, the patient 400 is already given acupuncture, lying on the bed 300, before a magnetic stimulus is applied by the wireless magnetic needle system.

The current generating unit 100 generates a current to be supplied to the Helmholtz coil 200. If necessary, the current generating unit 100 may include a modulator (not shown) and an amplifier (not shown).

An external magnetic field is required in order to apply a variety of magnetic stimuli to the human body of the patient 400 through the wireless magnetic needle. The external magnetic field is created by applying the current generated by the current generating unit 100 to the Helmholtz coil 200.

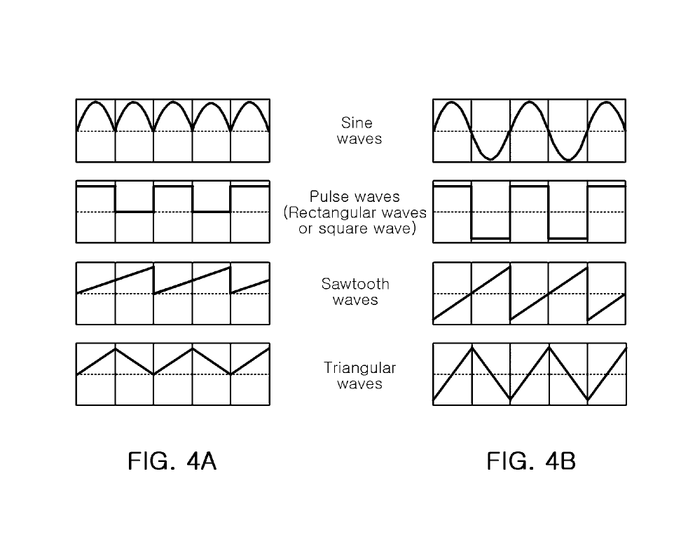

The current generated by the current generating unit 100 may be provided in various forms, as illustrated in FIGS. 4A and 4B. Herein, the waveform of the current may be provided efficiently according to the operational principle and the material of the soft-magnetic needle.

The Helmholtz coil 200 may be formed in various structures. In an exemplary embodiment of the present invention, the Helmholtz coil 200 includes the first coaxial coil 210 disposed over the bed 300 and the second coaxial coil 220 disposed under the bed 300.

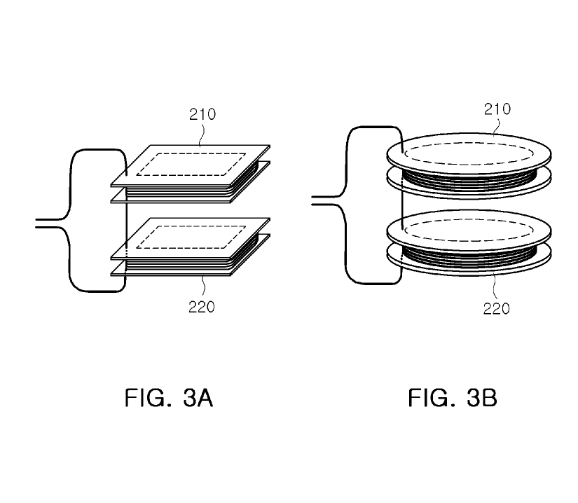

The first coaxial coil 210 and the second coaxial coil 220 of the Helmholtz coil 200 may have various shapes. In an exemplary embodiment of the present invention, the first coaxial coil 210 and the second coaxial coil 220 have a rectangular shape or a square shape as shown in FIG. 3A. In another exemplary embodiment of the present invention, the first coaxial coil 210 and the second coaxial coil 220 have a circular shape as shown in FIG. 3B.

In an exemplary embodiment of the Helmholtz coil 200, the first coaxial coil 210 located over the bed 300 is movable and the second coaxial coil 220 located under the bed 300 is fixed, as shown in FIG. 1.

After the patient 400 is given acupuncture lying on the bed 300, a current is supplied from the current generating unit 100 to the Helmholtz coil 200 to create a magnetic field between the first coaxial coil 210 and the second coaxial coil 220. Herein, the second coaxial coil 220 is fixed and the first coaxial coil 210 moves between the head and the leg of the patient 400, thereby applying magnetic stimuli thereto.

In a modified embodiment of the Helmholtz coil 200, the first coaxial coil 210 is fixed and the second coaxial coil 220 is movable.

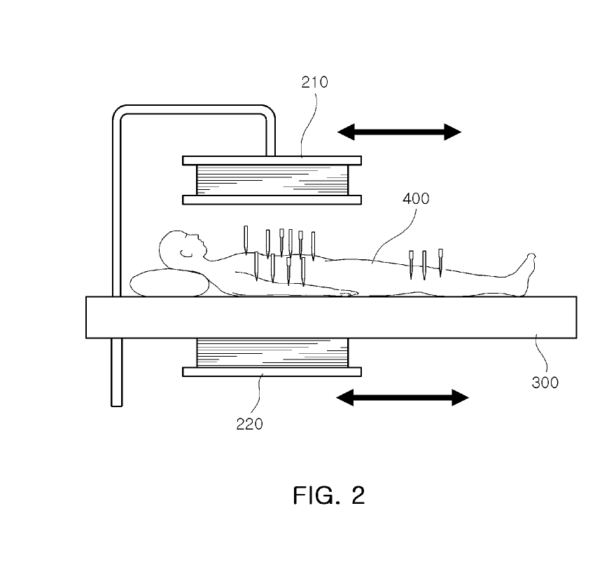

In another exemplary embodiment of the Helmholtz coil 200, both of the first coaxial coil 210 and the second coaxial coil 220 are movable as shown in FIG. 2.

FIG. 2 is a schematic view showing a wireless magnetic needle system for traditional oriental medicine according to another exemplary embodiment of the present invention.

Referring to FIG. 2, the first coaxial coil 210 located over the bed 300 and the second coaxial coil 220 located under the bed 300 move between the head and the leg of the patient 400, thereby applying magnetic stimuli thereto.

In the wireless magnetic needle system according to the present invention, the bed 300 and peripheral devices are formed of non-magnetic materials so that the magnetic field can be efficiently applied to the soft-magnetic needle.

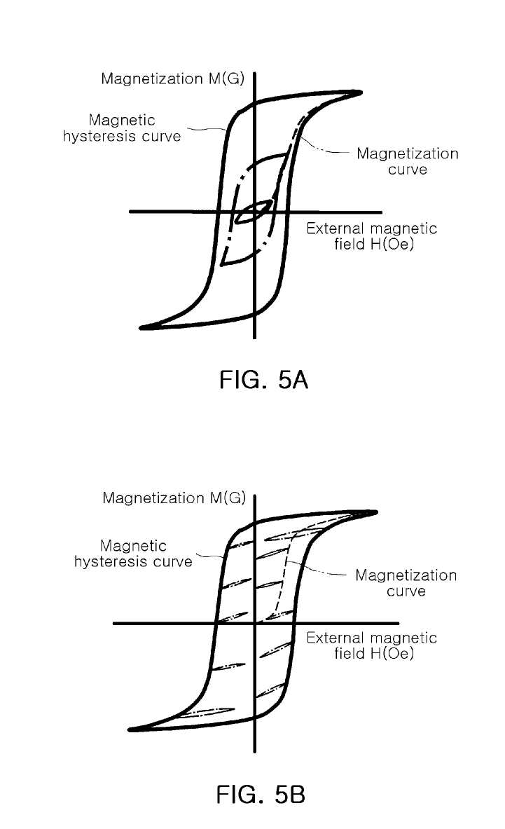

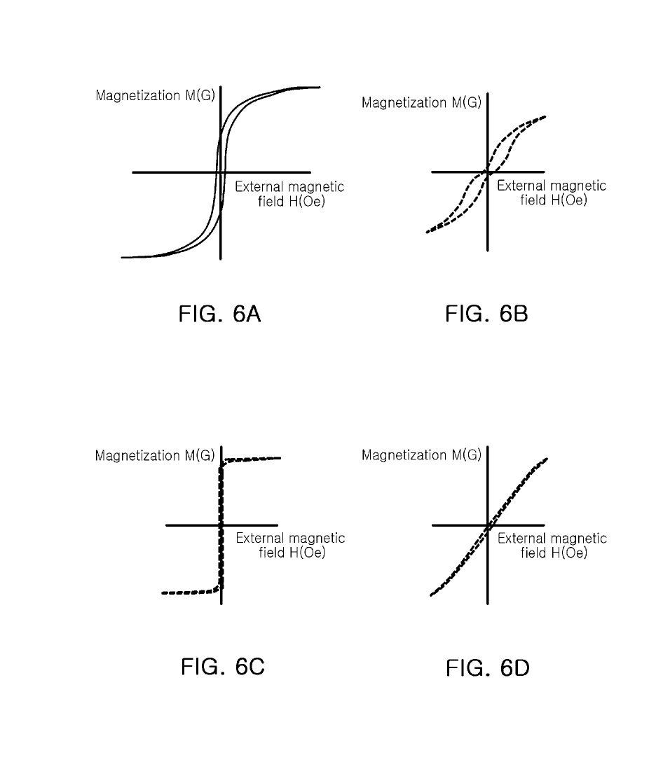

FIGS. 5A and 5B are graphs illustrating the operational characteristics of a soft-magnetic needle for traditional oriental medicine. FIGS. 6A to 6D are graphs illustrating the magnetic characteristics of a soft-magnetic needle for traditional oriental medicine.

Various currents formed as illustrated in FIGS. 4A and 4B are supplied to the Helmholtz coil 200 and are applied to the soft-magnetic needle. In this case, the operational characteristics of the soft-magnetic needle are determined according to magnetic hysteresis curves illustrated in FIGS. 5A and 5B.

In FIG. 5A, the axis of abscissas represents an external magnetic field and the axis of ordinates represents the reaction (magnetization) of a magnetic material with respect to the external magnetic field. As can be seen from FIG. 5A, when the external magnetic field increases over a certain level, the magnetization of the magnetic material saturates without change. At this point, when the external magnetic field is applied in the opposite direction, the magnetic material reacts along a path different from the original path. This phenomenon is called a magnetic hysteresis phenomenon.

When the external magnetic field applied in the opposite direction increases, the magnetization of the magnetic material saturates in the opposite direction. Thereafter, when the magnetic material is again magnetized in the original direction, it forms a completely closed curve that is symmetrical with respect to the origin. This curve is called a magnetic hysteresis curve.

A curve obtained in the original magnetization of the magnetic material is called a magnetization curve. The strength of the external magnetic field on the magnetic hysteresis curve, where the magnetization value becomes 0, is called a coercive force. In the present invention, the soft-magnetic material used as the material of the wireless magnetic needle for traditional oriental medicine means a magnetic material with a very-small coercive force. For reference, a hard-magnetic material (such as a permanent magnet) used in a conventional magnetic needle is a magnetic material with a very-large coercive force.

The magnetic hysteresis curve of FIG. 5A relates to the soft-magnetic needle for traditional oriental medicine. As can be seen from FIG. 5A, the magnetization value of the soft-magnetic material changes nonlinearly according to the strength of the external magnetic field but may be controlled in principle. Herein, minor hysteresis curves within the saturated magnetic hysteresis curves may be formed according to the time delay of the reaction of the magnetic material when an AC external magnetic field is rapidly applied to the soft-magnetic material or when the external magnetic field is not strong enough to saturate the magnetic material.

When the form of the current of the current generating unit 100 forming the external magnetic field is symmetrical bi-phase as illustrated in FIG. 4B, the magnetic polarity (N, S) of the soft-magnetic needle for traditional oriental medicine changes at various frequencies and its operational characteristics may be determined according to the magnetic hysteresis curve of FIG. 5A.

On the other hand, when the form of the current of the current generating unit 100 forming the external magnetic field is mono-phase as illustrated in FIG. 4A, the operational characteristics are determined according to the minor magnetic hysteresis curves illustrated in FIG. 5B. That is, in the case of a rectified sine wave, the external magnetic field increases from 0, decreases to 0 in the state of failing to reach a strength level sufficient to saturate the magnetic material, and increases again, and this process is repeated. In this case, the following two operational methods may be applied.

In the first operational method, as illustrated in FIG. 5B, the magnetization curve and the end point operate along the path of the minor magnetic hysteresis curve. In the second operational method, a magnetic field capable of saturating the magnetic material is applied once and then a weak mono-phase AC magnetic field is applied. In this case, it reacts along the minor hysteresis curve.

In this way, the operation of the wireless magnetic needle for traditional oriental medicine may vary, depending on the forms of the current generated by the current generating unit 100. Also, the operational characteristics may vary depending on the materials of the wireless magnetic needle for traditional oriental medicine.

FIG. 6A illustrates a magnetic hysteresis curve obtained from a general soft-magnetic material. This material may be converted into materials with characteristics illustrated in FIGS. 6B, 6C and 6D, through various fabrication (treatment) processes. In the case of FIG. 6C, because it may be completely saturated by a very-low external magnetic field, the polarity of the wireless magnetic needle for traditional oriental medicine may be converted by a fixed magnetic field and a magnetic stimulus may be applied to an acupuncture needle point. In the case of FIG. 6D, it reacts almost linearly with respect to the external magnetic field, and thus it is efficient in controlling the strength of the stimulus.

Another advantage of the wireless magnetic needle using the Helmholtz coil 200 is described below. Because the magnetic field generated by the pulse-type current applied by the current generating unit 100 can excite various harmonics for one pulse, it can apply various forms of magnetic stimuli and can implement infinite forms of magnetic stimuli by changing the width and cycle of the pulse.

Also, the AC magnetic field generated by the Helmholtz coil 200 induces a change of the magnetic field of the soft-magnetic needle to generate a micro current, thereby making it possible to achieve not only a magnetic stimulus but also an electrical stimulus at the acupuncture needle point.

The wireless magnetic needle, which is used on the patient 400 to apply a magnetic stimulus to the patient 400 through the wireless magnetic needle system for traditional oriental medicine, must have soft-magnetic characteristics as well as biocompatibility that are not harmful to the human body. Thus, the wireless magnetic needle is formed of a soft-magnetic material. For example, the wireless magnetic needles may be formed of an alloy of Cu, Mo and Cr based on Fe and Ni. A Co-based soft-magnetic material may be coated with gold or silver in order to provide a greater degree of safety.

As described above, the wireless magnetic needle system for traditional oriental medicine according to the present invention can wirelessly apply a magnetic stimulus having various strengths and forms of magnetic stimuli after acupuncture is performed on a patient according to a general method.

Also, the soft-magnetic material is used as the material of the wireless magnetic needle of the wireless magnetic needle system for traditional oriental medicine according to the present invention. Thus, the wireless magnetic needle for traditional oriental medicine according to the present invention can be fabricated in the same form as the conventional needle for traditional oriental medicine. Therefore, the wireless magnetic needle system for traditional oriental medicine according to the present invention is easy to fabricate.

Also, the wireless magnetic needle of the wireless magnetic needle system for traditional oriental medicine according to the present invention can be fabricated as a disposable type with a simple structure. Thus, the wireless magnetic needle of the wireless magnetic needle system for traditional oriental medicine according to the present invention can prevent possible cross-infection between patients.

While the present invention has been shown and described in connection with the exemplary embodiments, it will be apparent to those skilled in the art that modifications and variations can be made without departing from the spirit and scope of the invention as defined by the appended claims.

What is claimed is:

1. A wireless magnetic needle system for traditional oriental medicine, comprising:
a current generating unit generating a current; and
a Helmholtz coil creating a magnetic field by using the current generated by the current generating unit, the Helmholtz coil comprising,
a first coaxial coil disposed over a bed for a patient; and
a second coaxial coil disposed under the bed.

2. The wireless magnetic needle system of claim 1, further comprising:
a modulator modulating the current generated by the current generating unit; and
an amplifier amplifying the current modulated by the modulator.

3. The wireless magnetic needle system of claim 1, wherein the current generated by the current generating unit has a waveform of one of a rectangular pulse wave, a sine wave and a sawtooth wave.

4. The wireless magnetic needle system of claim 1, wherein the first coaxial coil or the second coaxial coil of the Helmholtz coil has a quadrangular shape or a circular shape.

5. The wireless magnetic needle system of claim 1, wherein the first coaxial coil of the Helmholtz coil is movable and the second coaxial coil of the Helmholtz coil is fixed.

6. The wireless magnetic needle system of claim 1, wherein both of the first coaxial coil and the second coaxial coil of the Helmholtz coil are movable.

7. A wireless magnetic needle for a wireless magnetic needle system for traditional oriental medicine that applies a current generated by a current generating unit to a Helmholtz coil to apply a magnetic stimulus to a needle being used for the acupuncture process on a patient, wherein the wireless magnetic needle is formed of a soft-magnetic material.

8. The wireless magnetic needle of claim 7, wherein the wireless magnetic needle is formed of an alloy including iron, nickel or cobalt.
